(19) United States
(12) Patent Application Publication
(10) Pub. No.: US 2021/0270981 A1
(43) Pub. Date: Sep. 2, 2021

(54) FULL-ANGLE COINCIDENCE PET DETECTOR AND METHOD

(71) Applicant: SHANDONG MADIC TECHNOLOGY CO., LTD., Linyi City, Shandong Province (CN)

(72) Inventor: Jiguo LIU, Linyi City, Shandong Province (CN)

(73) Assignee: SHANDONG MADIC TECHNOLOGY CO., LTD., Linyi City, Shandong Province (CN)

(21) Appl. No.: 17/053,535

(22) PCT Filed: Apr. 1, 2019
(86) PCT No.: PCT/CN2019/080785

§ 371 Date: Nov. 6, 2020

(30) Foreign Application Priority Data
May 7, 2018 (CN) 201810426783.8
May 7, 2018 (CN) 201810426785.7

(51) Int. Cl. G01T 1/29; G01T 1/20; A61B 6/03; A61B 6/00

(52) U.S. Cl. CPC G01T 1/2985; A61B 6/4266; A61B 6/037; G01T 1/20185

(57) ABSTRACT

A full-angle coincidence PET detector array, comprising the following components: a plurality of PET detection modules (2), wherein each of the PET detection modules (2) is composed of PET detection crystals (7), a photosensor array (5) and a light guide (6); and the plurality of PET detection modules (2) are adjacent to each other to form an integrally closed detection chamber. A full-angle coincidence PET detection method, comprising the following steps: 1) the step of assembling the detection chamber; 2) the step of placing a detection object; and 3) the step of acquiring an image. The cross-sectional area of all voids is smaller than the area of the smallest of the PET detection crystals (7) when the detection chamber is in a closed state; and the integrally closed detection chamber is of a cylindrical shape, a capsular shape, an ellipsoidal shape or a regular polygonal prism shape.

### TECHNICAL FIELD

The present disclosure relates to the technical field of PET detectors, and in particular to a PET detector that detects the arrangement of crystals, a full-angle coincidence PET detector and a full-angle coincidence detection method using the detector, none of which was ever seen in the related art.

### BACKGROUND

Positron Emission Tomography (PET) apparatuses are widely used in specificity imaging of animals and human bodies (hereinafter referred to as scanned object). In PET imaging, it is required to first inject a tracer labeled with a positron nuclide into the scanned object, and then image the distribution of the tracer in the scanned object. The imaging of the position labeled by the tracer has strong specificity, and dynamic imaging may be performed with a high degree of recognition.

Traditional PET apparatuses have insufficient axial depth of the detector and can only scan a limited local area at one time. If it is desired to obtain a PET image of the whole body of human, local scanned images of multiple (such as 8-10) beds must be spliced to obtain the image of whole body. There are two problems with this imaging method: first, the imaging speed is slow, each bed takes 1 to 5 minutes for a traditional human body PET apparatus, the axial field of view is about 20 cm, the whole-body imaging requires 8-10 beds and takes at least 8 minutes at one time, and additional calculation time is further required, which may reach 50 minutes for some apparatuses; second, one of the major advantages of PET is that dynamic information of the tracer can be obtained, but it is impossible for the detector with insufficient depth to obtain the dynamic information of the tracer on the whole body, and the images obtained at different beds cannot be spliced to obtain the dynamic information of the whole body; this is an impossible task for the traditional PET apparatus, which is shown in FIG. 1. Traditional detectors can only image a part of the region of interest, and the sensitivity of the generated image is also insufficient. For example, if the depth/length of a detector ring is 30 cm, it can actually only detect one part, such as the abdomen of the human body; moreover, due to the angle problem, most of the LOR photoelectrons are incident into an opening portion of the detector ring, and the sensitivity of the image obtained is only about 10%. It is impossible for the traditional PET apparatus, which is shown in FIG. 1, to greatly improve the sensitivity.

In special cases, in order to obtain the status of systemic drug metabolism, an axial field of view of extended PET apparatus has appeared in the related art. When the length/depth of the axial field of view exceeds or approaches the length of the scanned object, the whole-body dynamic imaging can be performed on the scanned object. For example, in Sci. Transl. Med, vol. 9, eaaf6169 (2017) 15 Mar. 2017 by Cherry et al., by axially extending the detector ring of the human body PET to 2 meters, dynamic imaging can be performed on the whole human body. However, the size of the PET detector ring of these whole-body imaging apparatuses is completely uniform in the entire axial direction, and only the length/depth of the detector is extended in the axial direction. The problem with such detector ring design is that the sensitivity in the scanning field of view is not uniform enough. The sensitivity is the highest in the middle of the overall detector. As the position moves from a center to both ends of the detector along an axis, the sensitivity drops rapidly, and drops to a very low level at the positions of the two ends of the detector, or even zero. FIG. 2 shows an extended PET apparatus in the related art. However, the PET detector ring of these whole body imaging devices only extends the length/depth of the detector in the axial direction. The problem with such detector ring design is that the sensitivity in the scanning field of view is not uniform enough. The sensitivity is the highest in the middle of the overall detector. As the position moves from a center to both ends of the detector along an axis, the sensitivity drops rapidly, and drops to a very low level at the positions of the two ends of the detector, or even zero. FIG. 2 shows an extended PET apparatus in the related art. Although the range that can be captured by one imaging is greatly increased in this way, the image obtained still has a big problem, that is, the sensitivity is uneven; moreover, the sensitivity is not only uneven, but there is still a huge gap between such a capture and an almost complete capture of LOR.

The reason for this phenomenon is that PET adopts a data acquisition method of coincidence detection. When 511 keV gamma rays are simultaneously detected on two exactly opposite detector crystals, this is called a true coincidence event. Only in this situation will the two gamma rays be taken as an effective positron event. Occurrence positions of this positron event are on a straight line between the two crystals, which are positions to be detected. This line is called line of reaction, hereinafter referred to as LOR.

FIG. 4 is a schematic diagram of the LORs of a PET detector in the related art. It can be clearly seen from a comparison between two occurrence positions in the figure that one position is at the center of the axial field of view of the detector, and the other position is not at the center of the axial field of view, but at the edge. Due to the difference in position, the probabilities of detecting LORs occurring from different positions differ greatly. For most LORs that occur from the center, they can be detected as long as they are not horizontal or nearly horizontal; for LORs occurring from the edge, only some LORs that are perpendicular or nearly perpendicular to the axial direction can be detected. The number of LORs that can be detected occurring from non-center positions is significantly lower than the number of LORs occurring from the center, which leads to the fact that the sensitivity becomes lower and lower as the occurrence position deviates from the LOR center. The sensitivity of any point in the PET field of view is determined by a solid angle covered by all LORs passing through the point. The larger the solid angle covered by the LORs is, the greater the sensitivity of the point will be. This relationship between the sensitivity and the position is shown in FIG. 3, which shows that the closer it is to the center of gravity, the higher the sensitivity will be; on the contrary, the sensitivity at the edge is very low.

The sensitivity of any point in the PET field of view is determined by a solid angle covered by all LORs passing through the point. The larger the solid angle covered by the LORs is, the greater the sensitivity of the point will be. This relationship between the sensitivity and the position is shown in FIG. 3, which shows that the closer it is to the center of gravity, the higher the sensitivity will be; on the contrary, the sensitivity at the edge is very low. It can be clearly seen from a comparison between two occurrence positions in FIG. 4 that one position is at the center of the axial field of view of the detector, and the other position is not at the center of the axial field of view, but at the edge. First, a large part of the LORs that occur from all the positions is not detected. Second, due to the difference in position, the probabilities of detecting LORs occurring from different positions differ greatly. For most LORs that occur from the center, they can be detected as long as they are not horizontal or nearly horizontal, and the detection rate reaches as high as 50% to 60%; for LORs occurring from the edge, only some LORs that are perpendicular or nearly perpendicular to the axial direction can be detected. If the tilt angle is slightly larger, one end of the LOR will be located outside the detector so that the true coincidence event cannot be detected. The number of LORs that can be detected occurring from non-center positions is significantly lower than the number of LORs occurring from the center, which leads to the fact that the sensitivity is missed at all the occurrence positions and the sensitivity becomes lower and lower as the occurrence position deviates from the LOR center.

This leads to, for example, that the sensitivities of the positions of the human head and feet are much lower than that of the abdomen in the center of the field of view during the whole-body PET scanning. This problem cannot be solved by simply extending the axial length of the detector. In other words, even if the length of the detector ring is 2 meters, when observing the dynamic image of the whole body, only the dynamic image near the abdomen meets the observation requirements, and the dynamic images near the head and feet are still of no confidence and cannot be applied. It still needs 2-3 times of splicing to obtain a better whole-body image. The detector ring is greatly lengthened and the cost of the instrument is greatly increased. However, it can be seen from the above analysis that a whole-body image or a whole-body dynamic image cannot be well obtained at one time. The closer it is to the head and feet, the lower the confidence of the obtained image data will be, and the problem of one-time whole-body imaging or one-time dynamic imaging is not fundamentally solved. From the above analysis, it can be seen that a whole-body image or a whole-body dynamic image cannot be well obtained at one time by simply lengthening the detector ring. The closer it is to the head and feet, the lower the confidence of the obtained image data will be, and the problem of one-time whole-body imaging or one-time dynamic imaging is not fundamentally solved. As for the problem of how to quickly generate a whole-body image at one time in the way of nearly full capture of LORs, it has not been realized in the related art, nor meaningful explorations have been made for this problem in the related art.

### SUMMARY

A first object of the present disclosure is to solve the problem of low sensitivity at the edge of the PET detector ring in the related art, and to provide a perfect PET detector solution for this situation where there is no effective solution yet. With this arrangement, the problem that a credible whole-body image cannot be obtained at one time despite constant increase of the length/depth of the detector ring can be solved. This solution to the problem has not yet appeared in the relate art, and even the problem of sensitivity defect has not yet been clearly raised in the related art. In the related art, it is generally believed that the whole-body image can be obtained by lengthening the director ring, but it has never been thought that such a whole-body image is not suitable for use and does not meet the requirements. A second object of the present disclosure is to solve the problem in the related art that LORs are largely lost during the capture and cannot be almost completely captured at one time to generate a whole-body image with high sensitivity at one time. In view of this situation where there is no effective solution, a perfect PET detection method is provided. With this arrangement, the problem that a credible whole-body image cannot be obtained at one time despite constant increase of the length/depth of the detector ring can be solved. This solution to the problem has not yet appeared in the relate art, and even the problem of sensitivity defect has not yet been clearly raised in the related art. In the related art, it is generally believed that the whole-body image can be obtained by lengthening the director ring, but it has never been thought that such a whole-body image is not suitable for use and does not meet the requirements. Especially when acquiring dynamic images, the dynamic images acquired by the extended PET detection ring near the two ends are still of no confidence.

A full-angle coincidence PET detector array is provided, which includes a plurality of PET detection modules, each of which is composed of a photoelectric sensor array and a light guide.

The plurality of PET detection modules are adjacent to each other to form an integrally closed detection cavity, and PET detection crystals are all arranged in a direction toward an interior of the cavity.

Each of the cross-sectional areas of all gaps of the detection cavity is smaller than the area of the smallest one of the aforementioned PET detection crystals.

In a full-angle coincidence PET detector array as described above, the full-angle coincidence PET detector array has a cylindrical shape and is composed of a barrel in the middle and two planar end caps at both ends.

The barrel is composed of a plurality of detection module rings closely arranged to form a cylindrical shape, and each of the detection module rings is composed of a certain number of detection modules arranged circumferentially into a ring shape in a crystal-inward manner.

The planar end cap is composed of a certain number of detection modules arranged in parallel into a disc shape in a crystal-inward manner, and an inner side surface of the planar end cap formed into an approximately circular shape has a size larger than a circular opening of the aforementioned barrel.

In a full-angle coincidence PET detector array as described above, the full-angle coincidence PET detector array has a capsule shape and is composed of a barrel in the middle and two concave curved end caps at both ends; the barrel is composed of a plurality of detection module rings closely arranged to form a cylindrical shape, and each of the detection module rings is composed of a certain number of detection modules arranged circumferentially into a ring shape in a crystal-inward manner; the concave curved end cap is composed of a certain number of detection modules arranged in a certain curvature in a crystal-inwardly-concave manner, and the cross section of the concave curved end cap perpendicular to an axis of the barrel is larger than a circular opening of the barrel.

In a full-angle coincidence PET detector array as described above, the concave curved end cap is specifically one of the following three situations: a hemispherical end cap, a less-than-half ellipsoidal end cap or a less-than-half spherical crown-shaped end cap.

In a full-angle coincidence PET detector array as described above, the full-angle coincidence PET detector array has an ellipsoid shape with a>b=c, and is composed of two upper and lower hemi-ellipsoids or two left and right hemi-ellipsoids, or composed of two left and right hemi-ellipsoids with the barrel sandwiched therebetween; the upper and lower hemi-ellipsoids are mirror-symmetrical, and the left and right hemi-ellipsoids are mirror-symmetrical; the barrel is composed of a plurality of detection module rings closely arranged to form a cylindrical shape or a shape of truncated ellipsoid in the middle; each of the detection module rings is composed of a certain number of detection modules arranged circumferentially into a ring shape in a crystal-inward manner.

In a full-angle coincidence PET detector array as described above, the full-angle coincidence PET detector array has a regular polygonal prism shape and is composed of a barrel in the middle and two planar end caps at both ends; the barrel is composed of a plurality of detection module rings closely arranged to form a regular polygonal prism shape, and each of the detection module rings is composed of a certain number of detection modules arranged circumferentially into a regular polygon shape in a crystal-inward manner; the planar end cap is composed of a certain number of detection modules arranged in parallel into a disc shape in a crystal-inward manner, and an inner side surface of the planar end cap formed into an approximately circular shape has a size larger than a regular polygon opening of the aforementioned barrel.

A coincidence circuit is connected between every two PET detection modules; each of the PET detection modules has the following specific structure: a detector housing is wrapped on the outside, a photoelectric sensor array is disposed outwardly, and a PET detection crystal is disposed inwardly. A light guide is disposed between the photoelectric sensor array and the PET detection crystal. The light guide is tightly coupled with both the photoelectric sensor array and the PET detection crystal; the PET detection crystal is a scintillation crystal.

The scintillation crystal is composed of a crystal strip array, or is composed of one or more crystal blocks; the material of the scintillation crystal is selected from one or more of bismuth germanate (BGO) crystals, sodium iodide (NaI) crystals, NaI(Tl) single crystals, lutetium silicate (LSO) crystals, gadolinium silicate (GSO) crystals and yttrium lutetium silicate (LYSO).

Spacers made of high atomic number substance are installed between all the detection module rings, or spacers made of high atomic number substance are installed between some of the detection module rings, or no spacers are installed between all the detection module rings; the high atomic number substance is lead or tungsten; the regular polygonal prism is a regular hexagonal prism or a regular octagonal prism, and the regular polygon is a regular hexagon or a regular octagon.

The crystal strip array is composed of a plurality of crystal strips; and each of the one or more crystal blocks is composed of one or more integrally cut crystals.

A full-angle coincidence PET detection method includes the following steps: 1) a detection cavity assembly step: in which a plurality of PET detection modules are adjacent to each other to form an integrally closed detection cavity, wherein each of the PET detection modules is composed of a PET detection crystal, a photoelectric sensor array and a light guide, and the PET detection crystals are all arranged in a direction toward an interior of the cavity; 2) a detection object placement step: in which the detection cavity is opened by opening one end of the detection cavity or opening the detection cavity up and down or separating the detection cavity left and right, and a detection object is placed therein; and 3) an image acquisition step: in which the detection cavity is closed, and PET detection is performed while keeping the integrally closed state so that all static images or all dynamic images of the detection object in the detection cavity are obtained at one time.

The integrally closed state specifically means that each of the cross-sectional areas of all gaps of the detection cavity in the closed state is smaller than the area of the smallest one of the aforementioned PET detection crystals; the integrally closed detection cavity has one of the following shapes: cylindrical shape; capsule shape; ellipsoid shape; and regular polygonal prism shape. In step (2), the detection cavity is divided into two halves up and down or left and right. Each of the two halves of the detection cavity has a support structure to support the two halves of the detection cavity respectively. The opening and closing of the left and right halves of the detection cavity are realized by a linear guide rail located below. The opening and closing of the upper and lower halves of the detection cavity are realized by a vertical linear guide rail on the side; the linear guide rail is a linear guide rail for the movement of a scanning bed.

In the full-angle detection method as described above, when the integrally closed detection cavity has a cylindrical shape, it is composed of a barrel in the middle and two planar end caps at both ends; the barrel is composed of a plurality of detection module rings closely arranged to form a cylindrical shape, and each of the detection module rings is composed of a certain number of detection modules arranged circumferentially into a ring shape in a crystal-inward manner; the planar end cap is composed of a certain number of detection modules arranged in parallel into a disc shape in a crystal-inward manner, and an inner side surface of the planar end cap formed into an approximately circular shape has a size larger than a circular opening of the aforementioned barrel. When the integrally closed detection cavity has a cylindrical shape, the middle barrel is placed with the axis being horizontal, and the detection cavity has a housing outside. The housing is composed of a barrel housing on an outer surface of the barrel, and end cap housings on outer surfaces of the two planar end caps. Each of the two planar end cap housings is connected with the barrel housing by one or more hinges or coupling heads, so as to form an integrally closed detection cavity when closed; moreover, one or more fixation buckle devices are also included for closing the detection cavity.

When the integrally closed detection cavity has a capsule shape, it is composed of a barrel in the middle and two concave curved end caps at both ends; the barrel is composed of a plurality of detection module rings closely arranged to form a cylindrical shape, and each of the detection module rings is composed of a certain number of detection modules arranged circumferentially into a ring shape in a crystal-inward manner; the concave curved end cap is composed of a certain number of detection modules arranged in a certain curvature in a crystal-inwardly-directed manner, and the cross section of the concave curved end cap perpendicular to an axis of the barrel is larger than a circular opening of the barrel. When the integrally closed detection cavity has a capsule shape, the middle barrel is placed with the axis being horizontal, and the detection cavity has a housing outside. The housing is composed of a barrel housing on an outer surface of the barrel, and end cap housings on outer surfaces of the two concave curved end caps. Each of the two concave curved end cap housings is connected with the barrel housing by one or more hinges or coupling heads, so as to form an integrally closed detection cavity when closed; moreover, one or more fixation buckle devices are also included for closing the detection cavity. The concave curved end cap is one of the following three situations: a hemispherical end cap, a less-than-half ellipsoidal end cap or a less-than-half spherical crown-shaped end cap.

When the integrally closed detection cavity has an ellipsoid shape, a>b=c, and it is composed of two upper and lower hemi-ellipsoids or two left and right hemi-ellipsoids, or composed of two left and right hemi-ellipsoids with the barrel sandwiched therebetween; the upper and lower hemi-ellipsoids are mirror-symmetrical, and the left and right hemi-ellipsoids are mirror-symmetrical; the barrel is composed of a plurality of detection module rings closely arranged to form a cylindrical shape; each of the detection module rings is composed of a certain number of detection modules arranged circumferentially into a ring shape in a crystal-inward manner.

When the integrally closed detection cavity has an ellipsoid shape and the barrel is sandwiched in the middle, the middle barrel is placed with the axis being horizontal, and the detection cavity has a housing outside. The housing is composed of a barrel housing on an outer surface of the barrel, and two hemi-ellipsoid housings on outer surfaces of the two left and right hemi-ellipsoids. Each of the two hemi-ellipsoid housings is connected with the barrel housing by one or more hinges or coupling heads, so as to form an integrally closed detection cavity when closed; moreover, one or more fixation buckle devices are also included for closing the detection cavity. The barrel sandwiched in the middle of the ellipsoid-shaped detection cavity is a cylindrical barrel or a middle barrel cut from an ellipsoid that satisfies a>b=c.

When the integrally closed detection cavity has an ellipsoid shape and is composed of two upper and lower hemi-ellipsoids or two left and right hemi-ellipsoids, the detection cavity has a housing outside. The housing is composed of two upper and lower hemi-ellipsoid housings or two left and right hemi-ellipsoid housings that fit the two upper and lower hemi-ellipsoids or two left and right hemi-ellipsoids. The two upper and lower hemi-ellipsoid housings or the two left and right hemi-ellipsoid housings are each connected with the barrel housing by one or more hinges or coupling heads, so as to form an integrally closed detection cavity when closed; moreover, one or more fixation buckle devices are also included for closing the detection cavity.

When the integrally closed detection cavity has a regular polygonal prism shape, it is composed of a barrel in the middle and two planar end caps at both ends; the barrel is composed of a plurality of detection module rings closely arranged to form a regular polygonal prism shape, and each of the detection module rings is composed of a certain number of detection modules arranged circumferentially into a regular polygon shape in a crystal-inward manner; the planar end cap is composed of a certain number of detection modules arranged in parallel into a disc shape in a crystal-inward manner, and an inner side surface of the planar end cap formed into an approximately circular shape has a size larger than a regular polygon opening of the aforementioned barrel. When the integrally closed detection cavity has a regular polygonal prism shape, the middle barrel is placed with the axis being horizontal, and the detection cavity has a housing outside. The housing is composed of a barrel housing on an outer surface of the barrel, and end cap housings on outer surfaces of the two planar end caps. Each of the two end cap housings is connected with the barrel housing by one or more hinges or coupling heads, so as to form an integrally closed detection cavity when closed; moreover, one or more fixation buckle devices are also included for closing the detection cavity.

In the full-angle coincidence PET detection method as described above, a coincidence circuit is connected between every two PET detection modules; each of the PET detection modules has the following specific structure: a detector housing is wrapped on the outside, a photoelectric sensor array is disposed outwardly, and a PET detection crystal is disposed inwardly. A light guide is disposed between the photoelectric sensor array and the PET detection crystal. The light guide is tightly coupled with both the photoelectric sensor array and the PET detection crystal; the material of the PET detection crystal is a scintillation crystal, and the scintillation crystal is composed of one or more crystal blocks.

The PET detection crystal is selected from one or more of bismuth germanate (BGO) crystals, sodium iodide (NaI) crystals, NaI(Tl) single crystals, lutetium silicate (LSO) crystals, gadolinium silicate (GSO) crystals and yttrium lutetium silicate (LYSO). The crystal block is specifically a crystal strip array composed of a plurality of crystal strips, or is composed of one or more integrally cut crystals. Spacers made of high atomic number substance are installed between all the detection module rings, or spacers made of high atomic number substance are installed between some of the detection module rings, or no spacers are installed between all the detection module rings; the high atomic number substance is lead or tungsten; the regular polygonal prism is a regular hexagonal prism or a regular octagonal prism, and the regular polygon is a regular hexagon or a regular octagon.

In the full-angle coincidence PET detection method as described above, the crystal strip array is composed of a plurality of crystal strips; and each of the one or more crystal blocks is composed of one or more integrally cut crystals.

When the integrally closed detection cavity has a capsule shape, the specific configuration of the detection cavity is as follows: the detection cavity is divided into two left and right halves, and the two left and right halves of the detection cavity have a left support structure and a right support structure respectively for supporting the two left and right halves of the detection cavity; the two left and right halves of the detection cavity are opened and closed through a linear guide rail located below; the linear guide rail is a linear guide rail for the movement of a scanning bed, a pad block for adjusting the height of the guide rail is located below the linear guide rail, and a bed assembly above the guide rail can move along the guide rail as a whole; the scanning bed can have a scanning bed support, and since the scanning bed support needs a space, part of the PET detection modules can be removed.

In the step (2), the detection cavity is opened in the form of left and right separation; specifically, the support structures (1) for two left and right halves of the detection cavity drive the two left and right halves of the detection cavity to be separated along the guide rail (2) to the left and right; placing the detection object in the step (2) is to transfer the detection object to a suitable position on the scanning bed; closing the detection cavity in the step (3) means that the scanning bed and the scanning bed support (5) move to a scanning position along the scanning bed by means of the linear guide rail (3) and that the two left and right halves of the detection cavity are closed; in the step (3), the time of flight method is used to screen LORs of the true coincidence events during the calculation; after the step (3) is completed, the two left and right halves of the detection cavity are separated along the linear guide rail to the left and right, the scanning bed moves out of the scanning position, the detection object is replaced, and steps (1)-(3) are repeated.

The present disclosure has the following two main advantages. First, it completely solves the problem of obtaining whole-body image and whole-body dynamic image at one time. With the detector of the present disclosure, almost all LORs of the true coincidence events can be captured instantly. It fundamentally ensures the success rate of one-time imaging. Second, it completely solves the sensitivity problem of occurrence event capture. For example, only from the perspective of lengthening the detector, if it is desired to capture the occurrence positions on a more than 1-meter long human body with high sensitivity, the length of the detector may need to be 4 meters long to enable the sensitivity of the whole-body capture to meet the requirements. This is very uneconomical, since crystals such as bismuth germanate are expensive. The method of the present disclosure requires less materials than a 4-meter-long detector ring, but achieves a better effect. The sensitivities of nearly all occurrence positions are almost the same. This is something that no one has thought of and no one can achieve in the related art.

### BRIEF DESCRIPTION OF THE DRAWINGS

The accompanying drawings required to be used in the description of the embodiments of the present disclosure or the related art are described briefly below, so that the technical solutions according to the embodiments of the present disclosure or according to the related art will become clearer. It is apparent that the accompanying drawings in the following description show only some embodiments of the present disclosure. For those skilled in the art, other accompanying drawings may also be obtained according to these drawings provided, without any creative work.

FIG. 1 is a schematic diagram of a conventional PET detector ring and an object to be detected in the related art;

FIG. 2 is a schematic diagram of an axially lengthened detector ring that has appeared in recent years and an object to be measured;

FIG. 3 is a schematic diagram showing different sensitivities of the axially lengthened detector ring to various internal parts;

FIG. 4 is a schematic diagram showing that the LORs of different detection points can be captured in the axially lengthened detector ring;

FIG. 5 is a schematic diagram of a detection cavity composed of a barrel in the middle and two planar end caps at both ends;

FIG. 6 is a schematic diagram of a detection cavity composed of a barrel in the middle and two concave curved end caps at both ends;

FIG. 7 is a schematic diagram of a closed detection cavity having an entire ellipsoid shape;

FIG. 8 is a schematic diagram of a PET detection module with part of the housing cut away;

FIG. 9 is a schematic diagram showing how to detect in the two left and right halves of the detection cavity; and

FIG. 10 is a schematic diagram of a detection cavity having a capsule shape and composed of a body of a cylindrical barrel and two hemispherical end caps.

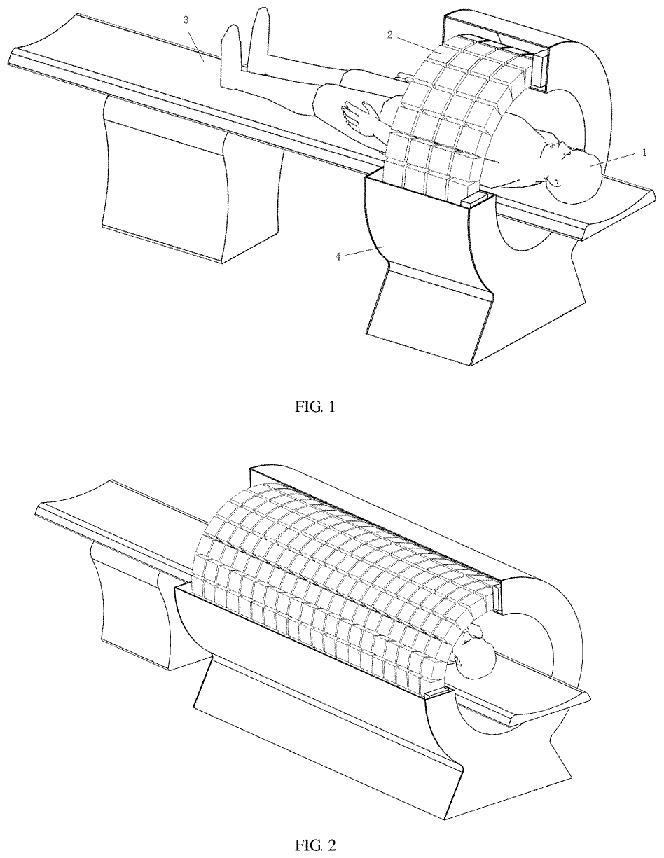

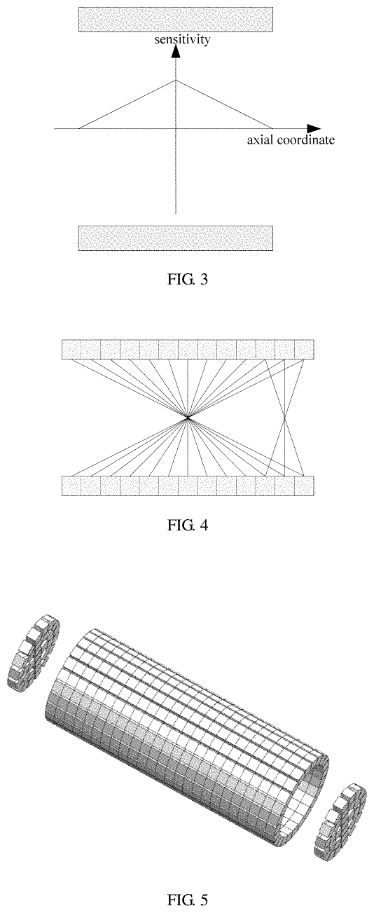

The devices corresponding to the reference signs are: 1: detection object; 2: PET detection module; 3: bracket; 4: base; 5: photoelectric sensor array; 6: light guide; 7: PET detection crystal; 8: left and right halves of the detection cavity; 9: left and right support structures; 10: linear guide rail; 11: linear guide rail for movement of scanning bed; 12: pad block; 13: scanning bed body; 14: scanning bed support; 15: triangular support part; 16: cylindrical support part.

### DETAILED DESCRIPTION

Preferred embodiments of the present disclosure will be described in detail below with reference to the accompanying drawings, so that the advantages and features of the present disclosure can be more easily understood by those skilled in the art, thereby making a clearer and definite definition of the scope of protection of the present disclosure.

A full-angle coincidence PET detector array includes a plurality of PET detection modules, each of which is composed of a PET detection crystal, a photoelectric sensor array and a light guide.

The plurality of PET detection modules are adjacent to each other to form an integrally closed detection cavity, and the PET detection crystals are all arranged in a direction toward an interior of the cavity. Herein, the PET detection crystals are all arranged in the direction toward the interior of the cavity, which means that the detection faces of the crystals are all arranged toward the interior to facilitate the detection of LORs. When the plurality of PET detection modules are adjacent to each other to form the integrally closed detection cavity, the specific forms of the various integrally closed detection cavities mentioned in the present application are effectively refined herein. In view that various convenient conditions of integral closing have been researched and trial-produced in the present application, it is reasonable and effective to refine the cavity of the present application into an integrally closed detection cavity.

Each of the cross-sectional areas of all gaps of the detection cavity is smaller than the area of the smallest one of the aforementioned PET detection crystals. Based on the shapes of the PET detection crystal, the photoelectric sensor array and the light guide, the PET detection modules of the present application all have a shape of rectangular parallelepiped or cuboid or a shape similar to rectangular parallelepipedor cuboid. It is necessary to reasonably arrange the position of each PET detection module so that there is no large gap exposed in the entire detection cavity, which would otherwise affect the realization of the technical solution of the present application. Each of the cross-sectional areas of all gaps of the detection cavity is smaller than the area of the smallest one of the aforementioned PET detection crystals. After such limitation, the generation of overly large gaps is avoided. The area of the smallest one of the aforementioned PET detection crystals may be one of 4*4 square centimeters, 5*5 square centimeters, 6*6 square centimeters, 7*7 square centimeters, 8*8 square centimeters, 9*9 square centimeters, and 10*10 square centimeters.

### First Embodiment

In a full-angle coincidence PET detector array as described above, specifically, the full-angle coincidence PET detector array has a cylindrical shape and is composed of a barrel in the middle and two planar end caps at both ends. The barrel is composed of a plurality of detection module rings closely arranged to form a cylindrical shape, and each of the detection module rings is composed of a certain number of detection modules arranged circumferentially into a ring shape in a crystal-inward manner. The planar end cap is composed of a certain number of detection modules arranged in parallel into a disc shape in a crystal-inward manner, and an inner side surface of the planar end cap formed into an approximately circular shape has a size larger than a circular opening of the aforementioned barrel.

This form of cylinder in the middle and planar end caps on both sides has a medium difficulty in processing. Since the PET detection modules of the present application all have a shape of rectangular parallelepiped or cuboid or a shape similar to rectangular parallelepipedor cuboid, the formed planar end caps can only be approximately circular, but generally not a complete circle, because the edge of the PET detection module is difficult to be shaped into a fan for matching. When the two planar end caps are in close contact, the three parts also form an integrally closed detection cavity. FIG. 5 is a schematic diagram of a detection cavity composed of a barrel in the middle and two planar end caps at both ends.

### Second Embodiment

In a full-angle coincidence PET detector array as described above, specifically, the full-angle coincidence PET detector array has a capsule shape and is composed of a barrel in the middle and two concave curved end caps at both ends; the barrel is composed of a plurality of detection module rings closely arranged to form a cylindrical shape, and each of the detection module rings is composed of a certain number of detection modules arranged circumferentially into a ring shape in a crystal-inward manner; the concave curved end cap is composed of a certain number of detection modules arranged in a certain curvature in a crystal-inwardly-concave manner, and the cross section of the concave curved end cap perpendicular to an axis of the barrel is larger than a circular opening of the barrel.

This form of cylinder in the middle and concave curved end caps on both sides has a medium difficulty in processing. The end caps require a three-dimensional design, especially because the PET detection modules of the present application all have a shape of rectangular parallelepiped or cuboid or a shape similar to rectangular parallelepipedor cuboid. A certain space is required to ensure the detection effect and avoid overly large gaps. The edges at which the concave curved end caps contact with the middle barrel are designed into a shape of a circular ring or an approximate circular ring in order to maintain close contact. When the two concave curved end caps are in close contact, the three parts also form an integrally closed detection cavity.

In a full-angle coincidence PET detector array as described above, the concave curved end cap is specifically one of the following three situations: a hemispherical end cap, a less-than-half ellipsoidal end cap or a less-than-half spherical crown-shaped end cap. Herein, the easiest way to design the concave curved end cap is the hemispherical end cap, but for the need to save materials, it may also be designed into a less-than-half spherical crown-shaped end cap. Out of the need for proper lengthening, it may also be designed into a less-than-half ellipsoidal end cap. For the needs of design, use and detection, the concave curved end caps herein are all designed to be symmetrical with respect to the central axis. The edges at which the end caps contact with the middle barrel are designed into a shape of a circular ring or an approximate circular ring in order to maintain close contact. When the two concave curved end caps are in close contact, the three parts also form an integrally closed detection cavity. FIG. 6 is a schematic diagram of a detection cavity composed of a barrel in the middle and two concave curved end caps at both ends. Although the special concave curved end caps are not further shown, there will be no designing and manufacturing obstacles to those skilled in the art when designing and implementing one of the hemispherical end cap, the less-than-half ellipsoidal end cap or the less-than-half spherical crown-shaped end cap.

### Third Embodiment

In a full-angle coincidence PET detector array as described above, the full-angle coincidence PET detector array has an ellipsoid shape with a>b=c, and is composed of two upper and lower hemi-ellipsoids or two left and right hemi-ellipsoids, or composed of two left and right hemi-ellipsoids with the barrel sandwiched therebetween; the upper and lower hemi-ellipsoids are mirror-symmetrical, and the left and right hemi-ellipsoids are mirror-symmetrical; the barrel is composed of a plurality of detection module rings closely arranged to form a cylindrical shape or a shape of truncated ellipsoid in the middle; each of the detection module rings is composed of a certain number of detection modules arranged circumferentially into a ring shape in a crystal-inward manner.

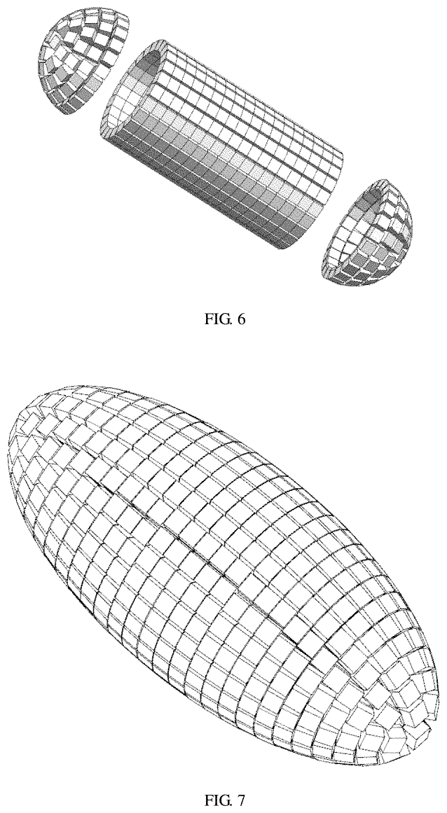

The overall ellipsoid shape seems to be currently a more economical way to save detection crystals, especially for long-strip-shaped detection objects. This design not only meets the need for economical use of detection crystals, but also can realize the integrally closed detection cavity at a lower cost. Of course, for design, production, and later LOR calculation and data collection considerations, it is best for this standard ellipsoidal cavity to be designed to be a>b=c. This axisymmetric ellipsoid is convenient for design, production and data collection. It is also possible to make an ellipsoid that is not axisymmetric. However, an ellipsoid that is not axisymmetric is not only complicated in design and production, but also it is difficult to locate and calculate later data. The ellipsoid that is not axisymmetric is basically difficult to apply in practice. In order to reduce the design difficulty in a certain sense, the ellipsoid shape may also be two left and right hemi-ellipsoids with the barrel sandwiched therebetween, and the barrel may have a cylindrical shape or a shape of ellipsoid in the middle with a different a>b=c truncated, which is also similar to the ellipsoid shape and which is also considered as falling within the implementations of the ellipsoid shape in the present application. Such a spliced design is convenient for modification over the related art, and the actual design and production difficulty is slightly lower, which is also a practically applicable implementation. FIG. 7 is a schematic diagram of a closed detection cavity having an entire ellipsoid shape.

### Fourth Embodiment

In a full-angle coincidence PET detector array as described above, the full-angle coincidence PET detector array has a regular polygonal prism shape and is composed of a barrel in the middle and two planar end caps at both ends; the barrel is composed of a plurality of detection module rings closely arranged to form a regular polygonal prism shape, and each of the detection module rings is composed of a certain number of detection modules arranged circumferentially into a regular polygon shape in a crystal-inward manner; the planar end cap is composed of a certain number of detection modules arranged in parallel into a disc shape in a crystal-inward manner, and an inner side surface of the planar end cap formed into an approximately circular shape has a size larger than a regular polygon opening of the aforementioned barrel.

The detection cavity in the form of a regular polygonal prism is very easy to design, manufacture and maintain. The disadvantage is that some detection crystals are wasted, and the combination and support at each edge requires certain auxiliary means. This form is easy to imagine, and no illustration is given herein.

For aesthetics or general design concepts, in the most common design, the regular polygonal prism is a regular hexagonal prism or a regular octagonal prism, and the regular polygon is a regular hexagon or a regular octagon.

### Fifth Embodiment

A coincidence circuit is connected between every two PET detection modules; each of the PET detection modules has the following specific structure: a detector housing is wrapped on the outside, a photoelectric sensor array is disposed outwardly, and a PET detection crystal is disposed inwardly. A light guide is disposed between the photoelectric sensor array and the PET detection crystal. The light guide is tightly coupled with both the photoelectric sensor array and the PET detection crystal; the PET detection crystal is a scintillation crystal.

The coincidence circuit is necessary for calculating the LOR, and can filter out the LORs of the true coincidence events most quickly. A portion of the detector housing that is located outside the PET detection crystal is designed as an opening, or the material used does not affect the collection of the positron emission signal.

The scintillation crystal is composed of a crystal strip array, and the crystal strip array is composed of a plurality of crystal strips; or the scintillation crystal is composed of one or more crystal blocks, each of which is composed of one or more integrally cut crystals. Two processing settings are proposed above, in which the crystal block method is simple in processing, and the crystal strip array method has a good coupling effect with the light guide and a faster response speed.

The material of the scintillation crystal is selected from one or more of bismuth germanate (BGO) crystals, sodium iodide (NaI) crystals, NaI(Tl) single crystals, lutetium silicate (LSO) crystals, gadolinium silicate (GSO) crystals and yttrium lutetium silicate (LYSO). After experimentation, all existing scintillation crystals can be used for the PET detection in the present application, and the actually available scintillation crystals are not limited to the crystal types actually listed above. Other available scintillation crystals can be used as the PET detection crystals in the present application.

Spacers made of high atomic number substance are installed between all the detection module rings, or spacers made of high atomic number substance are installed between some of the detection module rings, or no spacers are installed between all the detection module rings; the high atomic number substance is lead or tungsten. Herein, the present technical solution can also be implemented even if no spacer is installed at all. However, installing the spacer can appropriately reduce the crosstalk and the electromagnetic influence between the PET detection modules, which is a way that may be considered. Herein, the spacers may be all installed, or may be installed between some modules according to specific conditions and needs, but not installed in other positions, all of which are possible.

A full-angle coincidence PET detection method includes the following steps: 1) a detection cavity assembly step: in which a plurality of PET detection modules are adjacent to each other to form an integrally closed detection cavity, wherein each of the PET detection modules is composed of a PET detection crystal, a photoelectric sensor array and a light guide, and the PET detection crystals are all arranged in a direction toward an interior of the cavity; 2) a detection object placement step: in which the detection cavity is opened by opening one end of the detection cavity or opening the detection cavity up and down or separating the detection cavity left and right, and a detection object is placed therein; and 3) an image acquisition step: in which the detection cavity is closed, and PET detection is performed while keeping the integrally closed state so that all static images or all dynamic images of the detection object in the detection cavity are obtained at one time.

The plurality of PET detection modules are adjacent to each other to form an integrally closed detection cavity, and the PET detection crystals are all arranged in a direction toward an interior of the cavity. Herein, the PET detection crystals are all arranged in the direction toward the interior of the cavity, which means that the detection faces of the crystals are all arranged toward the interior to facilitate the detection of LORs. When the plurality of PET detection modules are adjacent to each other to form the integrally closed detection cavity, the specific forms of the various integrally closed detection cavities mentioned in the present application are effectively refined herein. In view that various convenient conditions of integral closing have been researched and trial-produced in the present application, it is reasonable and effective to refine the cavity of the present application into an integrally closed detection cavity.

The integrally closed state specifically means that each of the cross-sectional areas of all gaps of the detection cavity in the closed state is smaller than the area of the smallest one of the aforementioned PET detection crystals; the integrally closed detection cavity has one of the following shapes: cylindrical shape; capsule shape; ellipsoid shape; and regular polygonal prism shape.

Each of the cross-sectional areas of all gaps of the detection cavity is smaller than the area of the smallest one of the aforementioned PET detection crystals. Based on the shapes of the PET detection crystal, the photoelectric sensor array and the light guide, the PET detection modules of the present application all have a shape of rectangular parallelepiped or cuboid or a shape similar to rectangular parallelepipedor cuboid. It is necessary to reasonably arrange the position of each PET detection module so that there is no large gap exposed in the entire detection cavity, which would otherwise affect the realization of the technical solution of the present application. Each of the cross-sectional areas of all gaps of the detection cavity is smaller than the area of the smallest one of the aforementioned PET detection crystals. After such limitation, the generation of overly large gaps is avoided. The area of the smallest one of the aforementioned PET detection crystals may be one of 4*4 square centimeters, 5*5 square centimeters, 6*6 square centimeters, 7*7 square centimeters, 8*8 square centimeters, 9*9 square centimeters, and 10*10 square centimeters.

### Sixth Embodiment

In the full-angle detection method as described above, when the integrally closed detection cavity has a cylindrical shape, it is composed of a barrel in the middle and two planar end caps at both ends; the barrel is composed of a plurality of detection module rings closely arranged to form a cylindrical shape, and each of the detection module rings is composed of a certain number of detection modules arranged circumferentially into a ring shape in a crystal-inward manner; the planar end cap is composed of a certain number of detection modules arranged in parallel into a disc shape in a crystal-inward manner, and an inner side surface of the planar end cap formed into an approximately circular shape has a size larger than a circular opening of the aforementioned barrel. When the integrally closed detection cavity has a cylindrical shape, the middle barrel is placed with the axis being horizontal, and the detection cavity has a housing outside. The housing is composed of a barrel housing on an outer surface of the barrel, and end cap housings on outer surfaces of the two planar end caps. Each of the two planar end cap housings is connected with the barrel housing by one or more hinges or coupling heads, so as to form an integrally closed detection cavity when closed; moreover, one or more fixation buckle devices are also included for closing the detection cavity.

This form of cylinder in the middle and planar end caps on both sides has a medium difficulty in processing. Since the PET detection modules of the present application all have a shape of rectangular parallelepiped or cuboid or a shape similar to rectangular parallelepipedor cuboid, the formed planar end caps can only be approximately circular, but generally not a complete circle, because the edge of the PET detection module is difficult to be shaped into a fan for matching. When the two planar end caps are in close contact, the three parts also form an integrally closed detection cavity. FIG. 5 is a schematic diagram of a detection cavity composed of a barrel in the middle and two planar end caps at both ends.

### Seventh Embodiment

When the integrally closed detection cavity has a capsule shape, it is composed of a barrel in the middle and two concave curved end caps at both ends; the barrel is composed of a plurality of detection module rings closely arranged to form a cylindrical shape, and each of the detection module rings is composed of a certain number of detection modules arranged circumferentially into a ring shape in a crystal-inward manner; the concave curved end cap is composed of a certain number of detection modules arranged in a certain curvature in a crystal-inwardly-directed manner, and the cross section of the concave curved end cap perpendicular to an axis of the barrel is larger than a circular opening of the barrel. When the integrally closed detection cavity has a capsule shape, the middle barrel is placed with the axis being horizontal, and the detection cavity has a housing outside. The housing is composed of a barrel housing on an outer surface of the barrel, and end cap housings on outer surfaces of the two concave curved end caps. Each of the two concave curved end cap housings is connected with the barrel housing by one or more hinges or coupling heads, so as to form an integrally closed detection cavity when closed; moreover, one or more fixation buckle devices are also included for closing the detection cavity. The concave curved end cap is one of the following three situations: a hemispherical end cap, a less-than-half ellipsoidal end cap or a less-than-half spherical crown-shaped end cap.

This form of cylinder in the middle and concave curved end caps on both sides has a medium difficulty in processing. The end caps require a three-dimensional design, especially because the PET detection modules of the present application all have a shape of rectangular parallelepiped or cuboid or a shape similar to rectangular parallelepipedor cuboid. A certain space is required to ensure the detection effect and avoid overly large gaps. The edges at which the concave curved end caps contact with the middle barrel are designed into a shape of a circular ring or an approximate circular ring in order to maintain close contact. When the two concave curved end caps are in close contact, the three parts also form an integrally closed detection cavity.

In a full-angle coincidence PET detector array as described above, the concave curved end cap is specifically one of the following three situations: a hemispherical end cap, a less-than-half ellipsoidal end cap or a less-than-half spherical crown-shaped end cap. Herein, the easiest way to design the concave curved end cap is the hemispherical end cap, but for the need to save materials, it may also be designed into a less-than-half spherical crown-shaped end cap. Out of the need for proper lengthening, it may also be designed into a less-than-half ellipsoidal end cap. For the needs of design, use and detection, the concave curved end caps herein are all designed to be symmetrical with respect to the central axis. The edges at which the end caps contact with the middle barrel are designed into a shape of a circular ring or an approximate circular ring in order to maintain close contact. When the two concave curved end caps are in close contact, the three parts also form an integrally closed detection cavity. FIG. 6 is a schematic diagram of a detection cavity composed of a barrel in the middle and two concave curved end caps at both ends. Although the special concave curved end caps are not further shown, there will be no designing and manufacturing obstacles to those skilled in the art when designing and implementing one of the hemispherical end cap, the less-than-half ellipsoidal end cap or the less-than-half spherical crown-shaped end cap. FIG. 10 is a schematic diagram of a detection cavity having a capsule shape and composed of a body of a cylindrical barrel and two hemispherical end caps.

### Eighth Embodiment

When the integrally closed detection cavity has an ellipsoid shape, a>b=c, and it is composed of two upper and lower hemi-ellipsoids or two left and right hemi-ellipsoids, or composed of two left and right hemi-ellipsoids with the barrel sandwiched therebetween; the upper and lower hemi-ellipsoids are mirror-symmetrical, and the left and right hemi-ellipsoids are mirror-symmetrical; the barrel is composed of a plurality of detection module rings closely arranged to form a cylindrical shape; each of the detection module rings is composed of a certain number of detection modules arranged circumferentially into a ring shape in a crystal-inward manner.

When the integrally closed detection cavity has an ellipsoid shape and the barrel is sandwiched in the middle, the middle barrel is placed with the axis being horizontal, and the detection cavity has a housing outside. The housing is composed of a barrel housing on an outer surface of the barrel, and two hemi-ellipsoid housings on outer surfaces of the two left and right hemi-ellipsoids. Each of the two hemi-ellipsoid housings is connected with the barrel housing by one or more hinges or coupling heads, so as to form an integrally closed detection cavity when closed; moreover, one or more fixation buckle devices are also included for closing the detection cavity. The barrel sandwiched in the middle of the ellipsoid-shaped detection cavity is a cylindrical barrel or a middle barrel cut from an ellipsoid that satisfies a>b=c.

When the integrally closed detection cavity has an ellipsoid shape and is composed of two upper and lower hemi-ellipsoids or two left and right hemi-ellipsoids, the detection cavity has a housing outside. The housing is composed of two upper and lower hemi-ellipsoid housings or two left and right hemi-ellipsoid housings that fit the two upper and lower hemi-ellipsoids or two left and right hemi-ellipsoids. The two upper and lower hemi-ellipsoid housings or the two left and right hemi-ellipsoid housings are each connected with the barrel housing by one or more hinges or coupling heads, so as to form an integrally closed detection cavity when closed; moreover, one or more fixation buckle devices are also included for closing the detection cavity.

The overall ellipsoid shape seems to be currently a more economical way to save detection crystals, especially for long-strip-shaped detection objects. This design not only meets the need for economical use of detection crystals, but also can realize the integrally closed detection cavity at a lower cost. Of course, for design, production, and later LOR calculation and data collection considerations, it is best for this standard ellipsoidal cavity to be designed to be a>b=c. This axisymmetric ellipsoid is convenient for design, production and data collection. It is also possible to make an ellipsoid that is not axisymmetric. However, an ellipsoid that is not axisymmetric is not only complicated in design and production, but also it is difficult to locate and calculate later data. The ellipsoid that is not axisymmetric is basically difficult to apply in practice. In order to reduce the design difficulty in a certain sense, the ellipsoid shape may also be two left and right hemi-ellipsoids with the barrel sandwiched therebetween, and the barrel may have a cylindrical shape or a shape of ellipsoid in the middle with a different a>b=c truncated, which is also similar to the ellipsoid shape and which is also considered as falling within the implementations of the ellipsoid shape in the present application. Such a spliced design is convenient for modification over the related art, and the actual design and production difficulty is slightly lower, which is also a practically applicable implementation. FIG. 7 is a schematic diagram of a closed detection cavity having an entire ellipsoid shape.

### Ninth Embodiment

When the integrally closed detection cavity has a regular polygonal prism shape, it is composed of a barrel in the middle and two planar end caps at both ends; the barrel is composed of a plurality of detection module rings closely arranged to form a regular polygonal prism shape, and each of the detection module rings is composed of a certain number of detection modules arranged circumferentially into a regular polygon shape in a crystal-inward manner; the planar end cap is composed of a certain number of detection modules arranged in parallel into a disc shape in a crystal-inward manner, and an inner side surface of the planar end cap formed into an approximately circular shape has a size larger than a regular polygon opening of the aforementioned barrel. When the integrally closed detection cavity has a regular polygonal prism shape, the middle barrel is placed with the axis being horizontal, and the detection cavity has a housing outside. The housing is composed of a barrel housing on an outer surface of the barrel, and end cap housings on outer surfaces of the two planar end caps. Each of the two end cap housings is connected with the barrel housing by one or more hinges or coupling heads, so as to form an integrally closed detection cavity when closed; moreover, one or more fixation buckle devices are also included for closing the detection cavity.

The detection cavity in the form of a regular polygonal prism is very easy to design, manufacture and maintain. The disadvantage is that some detection crystals are wasted, and the combination and support at each edge requires certain auxiliary means. This form is easy to imagine, and no illustration is given herein. For aesthetics or general design concepts, in the most common design, the regular polygonal prism is a regular hexagonal prism or a regular octagonal prism, and the regular polygon is a regular hexagon or a regular octagon.

### Tenth Embodiment

In the full-angle coincidence PET detection method as described above, a coincidence circuit is connected between every two PET detection modules; each of the PET detection modules has the following specific structure: a detector housing is wrapped on the outside, a photoelectric sensor array is disposed outwardly, and a PET detection crystal is disposed inwardly. A light guide is disposed between the photoelectric sensor array and the PET detection crystal. The light guide is tightly coupled with both the photoelectric sensor array and the PET detection crystal; the material of the PET detection crystal is a scintillation crystal, and the scintillation crystal is composed of one or more crystal blocks.

The coincidence circuit is necessary for calculating the LOR, and can filter out the LORs of the true coincidence events most quickly. A portion of the detector housing that is located outside the PET detection crystal is designed as an opening, or the material used does not affect the collection of the positron emission signal.

The PET detection crystal is selected from one or more of bismuth germanate (BGO) crystals, sodium iodide (NaI) crystals, NaI(Tl) single crystals, lutetium silicate (LSO) crystals, gadolinium silicate (GSO) crystals and yttrium lutetium silicate (LYSO). The crystal block is specifically a crystal strip array composed of a plurality of crystal strips, or is composed of one or more integrally cut crystals. Spacers made of high atomic number substance are installed between all the detection module rings, or spacers made of high atomic number substance are installed between some of the detection module rings, or no spacers are installed between all the detection module rings; the high atomic number substance is lead or tungsten; the regular polygonal prism is a regular hexagonal prism or a regular octagonal prism, and the regular polygon is a regular hexagon or a regular octagon. Herein, the present technical solution can also be implemented even if no spacer is installed at all. However, installing the spacer can appropriately reduce the crosstalk and the electromagnetic influence between the PET detection modules, which is a way that may be considered. Herein, the spacers may be all installed, or may be installed between some modules according to specific conditions and needs, but not installed in other positions, all of which are possible.

In the full-angle coincidence PET detection method as described above, the crystal strip array is composed of a plurality of crystal strips; and each of the one or more crystal blocks is composed of one or more integrally cut crystals.

### Eleventh Embodiment

When the integrally closed detection cavity has a capsule shape, the specific configuration of the detection cavity is as follows: the detection cavity is divided into two left and right halves, and the two left and right halves of the detection cavity have a left support structure and a right support structure respectively for supporting the two left and right halves of the detection cavity; the two left and right halves of the detection cavity are opened and closed through a linear guide rail located below; the linear guide rail is a linear guide rail for the movement of a scanning bed, a pad block for adjusting the height of the guide rail is located below the linear guide rail, and a bed assembly above the guide rail can move along the guide rail as a whole; the scanning bed can have a scanning bed support, and since the scanning bed support needs a space, part of the PET detection modules can be removed.

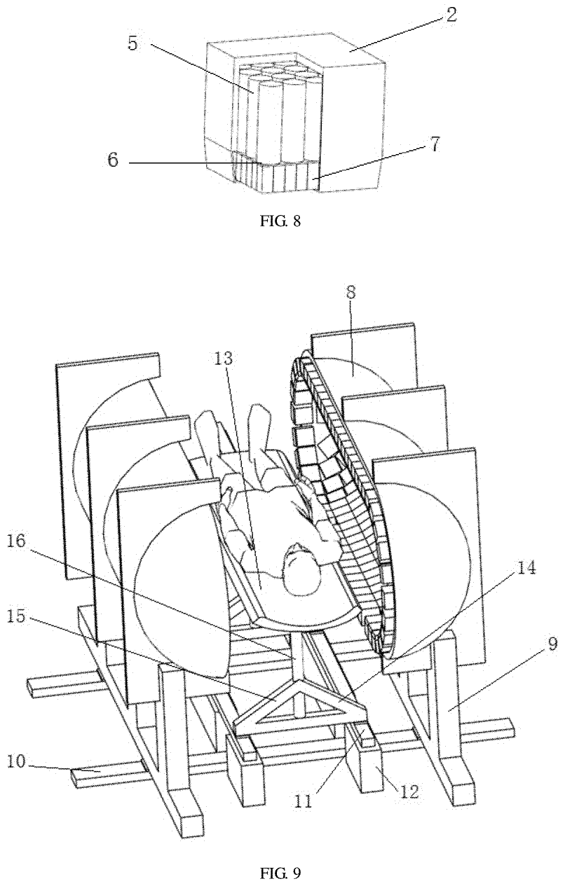

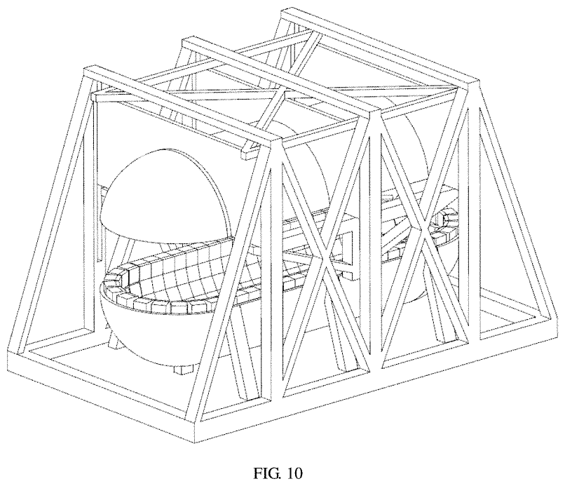

A detailed description of how the detection cavity of the capsule shape is divided into two halves is given below. As shown in FIG. 9, the left and right halves of the detection cavity 8 have left and right support structures 9 respectively for supporting the left and right detectors. Such separate support structures make it possible to separate the left and right halves of the detection cavity. The detection cavities on each side have a heavy weight, and it is impossible to expect them to be opened or closed by suspending or simply hanging or hoisting.

The linear guide rail 10 is used for the opening and closing of the detection cavity to the left and right, which allows the left and right halves of the detection cavity to be opened and closed accurately and to be automatically controlled. As shown in FIG. 9, there may be two linear guide rails for the detection cavity, which are parallel to each other.

In order to facilitate the movement and detection of the detection object, as shown in FIG. 9, a linear guide rail 11 for the movement of scanning bed is also provided. In order to adjust the detection height, a pad block 12 is located below the guide rail for adjusting the height of the guide rail, and the bed assembly above the guide rail can move along the guide rail for the movement of the scanning bed as a whole. There may also be two guide rails 11 for the movement of the scanning bed as shown in FIG. 9, which are parallel to each other. For the consideration of arrangement, the guide rail for the movement of the scanning bed is perpendicular to the above mentioned linear guide rail.

In order for the scanning bed body 13 to be suspended in the detection cavity, there is a scanning bed support 14 below the scanning bed body 13. The scanning bed support is connected to the scanning bed and the guide rail 11 for the movement of the scanning bed, and the scanning bed is made for easy detection. There are two front and rear scanning bed supports, and each support is composed of a triangular support portion 15 and a cylindrical support portion 16, as shown in FIG. 9.

In order to make room for the closed scanning bed support, the sizes of at least the corresponding 2-4 PET detection modules are reduced or 2-4 PET detection modules are removed. For electromagnetic shielding considerations, a plate with holes for shielding electromagnetic signals may be sleeved over the scanning bed support.

### Twelves Embodiment

For the aforementioned steps (1)-(3), 1) a detection cavity assembly step: in which a plurality of PET detection modules are adjacent to each other to form an integrally closed detection cavity, wherein each of the PET detection modules is composed of a PET detection crystal, a photoelectric sensor array and a light guide, and the PET detection crystals are all arranged in a direction toward an interior of the cavity; 2) a detection object placement step: in which the detection cavity is opened by opening one end of the detection cavity or opening the detection cavity up and down or separating the detection cavity left and right, and a detection object is placed therein; and 3) an image acquisition step: in which the detection cavity is closed, and PET detection is performed while keeping the integrally closed state so that all static images or all dynamic images of the detection object in the detection cavity are obtained at one time;

wherein in the step (1), an opening and closing test may be performed, and just after the power is turned on, a blank model of a non-living body is used for pre-scanning and pre-testing before the formal test; then in step (2), the PET detection module is in a standby state.

In the step (2), the detection cavity is opened in the form of left and right separation; specifically, the left and right support structures 9 drive the two left and right halves of the detection cavity 8 to be separated along the linear guide rail 10 to the left and right; placing the detection object in the step (2) is to transfer the detection object to a suitable position on the scanning bed body 13; and closing the detection cavity in the step (3) means that the scanning bed body 13 and the scanning bed support 14 move to a scanning position along the scanning bed by means of the guide rail 11 and that the two left and right halves of the detection cavity 8 are closed.

In the step (3), the time of flight method is used to screen LORs of the true coincidence events during the calculation.

After the step (3) is completed, the two left and right halves of the detection cavity 8 are separated along the linear guide rail 10 to the left and right, the scanning bed body 13 moves out of the scanning position, the detection object is replaced, and steps (1)-(3) are repeated.

The static image may be an image in any image format, and the dynamic image may be a continuous video stream in any format, or a series of images continuously acquired, which can be displayed and used for identification in a form similar to a CT image.

The basic working process of PET in the present application is as follows: (A) an accelerator is used to produce positron emission isotopes; organic compounds are labelled with positron emitters to become chemical tracers; first, an external nuclide radiation source is used to perform a transmission CT, and the transmission projection data is recorded; this set of data will be used for attenuation compensation later, and then the positron nuclide tracers are injected into the observation body; (B) the detector ring is used in vitro to detect the decay location of gamma photons; data is processed and images are reconstructed; and (C) the results are revealed. The detection steps (1)-(3) of the method of the present application are all within the aforementioned step (B), and step (A) and step (C) can be completed by various implementations in the related art, which are known to those skilled in the art.

The static image acquisition described in the present application is to count the detected annihilation events according to the LOR and store them in a projection data matrix, so that a set of static tomographic images can be reconstructed; the dynamic acquisition described in the present application is actually a set of successive static collections, which are used to observe the movement process of radiopharmaceuticals. In a specific imaging method, the PET detector detects positions of crystal strips on the ring hit by a pair of gamma photons respectively, which are obtained after conversion when the positrons in the same ring are annihilated, and these position signals are converted into electrical signals which, together with energy signals of the gamma photons and time information of the arrival time, are sent to a subsequent electronic front-end amplification and coincidence system. After that, the data of the two detector crystal strips hit by the selected true coincidence events are sent to a subsequent computer system via a computer interface. The computer counts the detected annihilation events according to the LOR and stores them in a projection data matrix (sinogram matrix) by layers. The data of each layer contains information about a specific angle, that is, sampling for each specific angle is the linear integral of all LOR values at this angle. In the projection data matrix (sinogram matrix) of each layer, the rows and columns of the matrix respectively represent the angle value and radioactivity sampling. Through mathematical operations and image reconstruction, images of selected layers in the object are reconstructed from these projection data, and tomographic images of the distribution of radiopharmaceuticals are reconstructed.

Herein, the image reconstruction can use two-dimensional reconstruction and three-dimensional reconstruction. Two-dimensional image reconstruction includes an analytical method and an iterative method. The analytical method is a back-projection method based on the central slice theorem, and the filtered back projection method (FBP) is commonly used. In the filtered back projection method, the projection data after Ram p filtering and low-pass window filtering at a certain angle is smeared back to the entire space according to the reverse direction of the projection direction, thereby obtaining a two-dimensional distribution. This method has the advantages of simple operation and easy clinical implementation, but its anti-noise ability is poor. When the collected data is relatively under-sampled and the heat source has a small size, it is often difficult to obtain satisfactory reconstructed images, and its quantitative accuracy is poor. The filtered back projection method can accurately reproduce the distribution of the tracers in the body when the projection data does not contain noise. This algorithm is often used for image reconstruction with less noise, such as head images. The iterative method is a numerical approximation algorithm; that is, starting from the initial value of the tomographic image, the estimated value of the image is repeatedly corrected to gradually approximate the true value of the tomographic image. Starting from a hypothetical initial image and using the iterative method, the theoretical projection value is compared with the measured projection value to find the optimal solution under the guidance of a certain optimization criterion. The solution process of the iterative method is: a. assuming an initial image; b. calculating the image projection; c. comparing with the measured projection value; d. calculating the correction coefficient and updating the initial image value; e. stopping the iteration when the stop rule is met; otherwise, taking the new reconstructed image as the initial image and starting from step b. Counting can take advantage of its high resolution in nuclear medicine imaging. The biggest disadvantage of the iterative method is the large amount of calculation and the slow calculation speed, which makes it difficult to meet the needs of clinical real-time reconstruction. Commonly used iterative methods in PET include Maximum Likelihood Expectation Maximization (MLEM) and Ordered Subset Expectation Maximization (OSEM) algorithms. OSEM is a fast iterative reconstruction algorithm that has been developed and perfected in recent years. It has the advantages of good spatial resolution, strong anti-noise ability, and faster speed than other iterative methods. It has been widely used in new nuclear medicine tomography apparatuses and is the main and practical iterative algorithm currently used in PET clinical application. The OSEM algorithm divides the projection data into n subsets. Only one subset is used to correct the projection data during each reconstruction, and the projection data is updated once when the image is reconstructed. In this way, the projection data is corrected once by all the subsets. As compared with the traditional iterative algorithm MLEM, the reconstructed image is refreshed by n times under approximately the same calculation time and the same amount of calculation, which greatly accelerates the image reconstruction speed and shortens the reconstruction time.

The effect of 3D reconstruction is better, but the data involved is massive. For example, for a detector with N detection rings, the data obtained by 3D scanning has N projection data matrices (sinogram matrix) perpendicular to the axial direction and N (N-1) projection data matrices (sinogram matrix) that are non-perpendicular to the axial direction, whereas 2D scanning mode only has 2N−1 matrix of data. For the collected three-dimensional data, the three-dimensional reconstruction method can be directly used. In order to increase the calculation speed and reduce the amount of calculation, the recombination method is usually used, namely the quasi-3D reconstruction method of PET, to recombine the three-dimensional data into two-dimensional data, and then the two-dimensional reconstruction method is used to obtains each tomographic image. The difficulty of 3D reconstruction lies in the incomplete volume data collection. The uncollected data must be estimated from the sinogram data of the 2D reconstructed tomographic image through a certain algorithm. The measured projection data and the estimated data are three-dimensionally reconstructed together through the filtered back projection method. One of the biggest advantages of the iterative method is that it can introduce constraints and condition factors related to the spatial geometry or the magnitude of the measured value according to the specific imaging conditions, such as operations for controlling iteration (e.g., the correction of spatial resolution non-uniformity, scattering attenuation correction, object geometry constraints, and smoothness constraints), thereby obtaining more accurate reconstructed images. With the improvement of computing power, 3D reconstruction has gradually become a general way.

Described above are only specific embodiments of the present disclosure, but the scope of protection of the present disclosure is not limited to this. Any change or replacement that can be contemplated without creative work should be covered within the scope of protection of the present disclosure. Therefore, the scope of protection of the present disclosure shall be accorded with the scope of the claims.

1. A full-angle coincidence PET detector array, comprising:
a plurality of PET detection modules, each of which is composed of a PET detection crystal, a photoelectric sensor array and a light guide;
wherein the plurality of PET detection modules are adjacent to each other to form an integrally closed detection cavity, and the PET detection crystals are all arranged in a direction toward an interior of the cavity; and
each of the cross-sectional areas of all gaps of the detection cavity is smaller than the area of the smallest one of the PET detection crystals.

2. The full-angle coincidence PET detector array according to claim 1, wherein:
the full-angle coincidence PET detector array has a cylindrical shape and is composed of a barrel in the middle and two planar end caps at both ends;
the barrel is composed of a plurality of detection module rings closely arranged to form a cylindrical shape, and each of the detection module rings is composed of a certain number of detection modules arranged circumferentially into a ring shape in a crystal-inward manner; and
the planar end cap is composed of a certain number of detection modules arranged in parallel into a disc shape in a crystal-inward manner, and an inner side surface of the planar end cap formed into an approximately circular shape has a size larger than a circular opening of the barrel.

3. The full-angle coincidence PET detector array according to claim 1, wherein:
the full-angle coincidence PET detector array has a capsule shape and is composed of a barrel in the middle and two concave curved end caps at both ends;
the barrel is composed of a plurality of detection module rings closely arranged to form a cylindrical shape, and each of the detection module rings is composed of a certain number of detection modules arranged circumferentially into a ring shape in a crystal-inward manner; and
the concave curved end cap is composed of a certain number of detection modules arranged in a certain curvature in a crystal-inwardly-concave manner, and the cross section of the concave curved end cap perpendicular to an axis of the barrel is larger than a circular opening of the barrel.

4. The full-angle coincidence PET detector array according to claim 1, wherein:
the concave curved end cap is specifically one of the following three situations: a hemispherical end cap, a less-than-half ellipsoidal end cap or a less-than-half spherical crown-shaped end cap.

5. The full-angle coincidence PET detector array according to claim 1, wherein:
the full-angle coincidence PET detector array has an ellipsoid shape with a>b=c, and is composed of two upper and lower hemi-ellipsoids or two left and right hemi-ellipsoids, or composed of two left and right hemi-ellipsoids with the barrel sandwiched therebetween; the upper and lower hemi-ellipsoids are mirror-symmetrical, and the left and right hemi-ellipsoids are mirror-symmetrical; and
the barrel is composed of a plurality of detection module rings closely arranged to form a cylindrical shape or a shape of truncated ellipsoid in the middle; each of the detection module rings is composed of a certain number of detection modules arranged circumferentially into a ring shape in a crystal-inward manner.

6. The full-angle coincidence PET detector array according to claim 1, wherein:
the full-angle coincidence PET detector array has a regular polygonal prism shape and is composed of a barrel in the middle and two planar end caps at both ends;
the barrel is composed of a plurality of detection module rings closely arranged to form a regular polygonal prism shape, and each of the detection module rings is composed of a certain number of detection modules arranged circumferentially into a regular polygon shape in a crystal-inward manner; and
the planar end cap is composed of a certain number of detection modules arranged in parallel into a disc shape in a crystal-inward manner, and an inner side surface of the planar end cap formed into an approximately circular shape has a size larger than a regular polygon opening of the barrel.

7. The full-angle coincidence PET detector array according to claim 2, wherein:
a coincidence circuit is connected between every two PET detection modules;
each of the PET detection modules has the following specific structure: a detector housing is wrapped on the outside, a photoelectric sensor array is disposed outwardly, and a PET detection crystal is disposed inwardly; a light guide is disposed between the photoelectric sensor array and the PET detection crystal; the light guide is tightly coupled with both the photoelectric sensor array and the PET detection crystal; and
the material of the PET detection crystal is a scintillation crystal, and the scintillation crystal is composed of one or more crystal blocks.

8. The full-angle coincidence PET detector array according to claim 7, wherein:
the crystal block is specifically a crystal strip array composed of a plurality of crystal strips, or is composed of one or more integrally cut crystals;
the material of the scintillation crystal is selected from one or more of bismuth germanate (BGO) crystals, sodium iodide (NaI) crystals, NaI(Tl) single crystals, lutetium silicate (LSO) crystals, gadolinium silicate (GSO) crystals and yttrium lutetium silicate (LYSO);
spacers made of high atomic number substance are installed between all the detection module rings, or spacers made of high atomic number substance are installed between some of the detection module rings, or no spacers are installed between all the detection module rings; and
the high atomic number substance is lead or tungsten; the regular polygonal prism is a regular hexagonal prism or a regular octagonal prism, and the regular polygon is a regular hexagon or a regular octagon.

9. The full-angle coincidence PET detector array according to claim 8, wherein:
the crystal strip array is composed of a plurality of crystal strips; and each of the one or more crystal blocks is composed of one or more integrally cut crystals.

10. A full-angle coincidence PET detection method, comprising the following steps:
1) a detection cavity assembly step: in which a plurality of PET detection modules are adjacent to each other to form an integrally closed detection cavity, wherein each of the PET detection modules is composed of a PET detection crystal, a photoelectric sensor array and a light guide, and the PET detection crystals are all arranged in a direction toward an interior of the cavity;
2) a detection object placement step: in which the detection cavity is opened by opening one end of the detection cavity or opening the detection cavity up and down or separating the detection cavity left and right, and a detection object is placed therein; and
3) an image acquisition step: in which the detection cavity is closed, and PET detection is performed while keeping the integrally closed state so that all static images or all dynamic images of the detection object in the detection cavity are obtained at one time.

11. The full-angle coincidence PET detection method according to claim 10, wherein:
Each of the integrally closed state specifically means that the cross-sectional areas of all gaps of the detection cavity in the closed state is smaller than the area of the smallest one of the PET detection crystals;
the integrally closed detection cavity has one of the following shapes: cylindrical shape; capsule shape; ellipsoid shape; and regular polygonal prism shape;
in step (2), the detection cavity is divided into two halves up and down or left and right; each of the two halves of the detection cavity has a support structure to support the two halves of the detection cavity respectively;
the opening and closing of the left and right halves of the detection cavity are realized by a linear guide rail located below, and the opening and closing of the upper and lower halves of the detection cavity are realized by a vertical linear guide rail on the side; and
the linear guide rail is a linear guide rail for the movement of a scanning bed.

12. The full-angle coincidence PET detection method according to claim 11, wherein:
when the integrally closed detection cavity has a cylindrical shape, it is composed of a barrel in the middle and two planar end caps at both ends; the barrel is composed of a plurality of detection module rings closely arranged to form a cylindrical shape, and each of the detection module rings is composed of a certain number of detection modules arranged circumferentially into a ring shape in a crystal-inward manner; the planar end cap is composed of a certain number of detection modules arranged in parallel into a disc shape in a crystal-inward manner, and an inner side surface of the planar end cap formed into an approximately circular shape has a size larger than a circular opening of the barrel;
when the integrally closed detection cavity has a capsule shape, it is composed of a barrel in the middle and two concave curved end caps at both ends; the barrel is composed of a plurality of detection module rings closely arranged to form a cylindrical shape, and each of the detection module rings is composed of a certain number of detection modules arranged circumferentially into a ring shape in a crystal-inward manner; the concave curved end cap is composed of a certain number of detection modules arranged in a certain curvature in a crystal-inwardly-directed manner, and the cross section of the concave curved end cap perpendicular to an axis of the barrel is larger than a circular opening of the barrel;
when the integrally closed detection cavity has an ellipsoid shape, a>b=c, and it is composed of two upper and lower hemi-ellipsoids or two left and right hemi-ellipsoids, or composed of two left and right hemi-ellipsoids with the barrel sandwiched therebetween; the upper and lower hemi-ellipsoids are mirror-symmetrical, and the left and right hemi-ellipsoids are mirror-symmetrical; the barrel is composed of a plurality of detection module rings closely arranged to form a cylindrical shape; each of the detection module rings is composed of a certain number of detection modules arranged circumferentially into a ring shape in a crystal-inward manner; and
when the integrally closed detection cavity has a regular polygonal prism shape, it is composed of a barrel in the middle and two planar end caps at both ends; the barrel is composed of a plurality of detection module rings closely arranged to form a regular polygonal prism shape, and each of the detection module rings is composed of a certain number of detection modules arranged circumferentially into a regular polygon shape in a crystal-inward manner; the planar end cap is composed of a certain number of detection modules arranged in parallel into a disc shape in a crystal-inward manner, and an inner side surface of the planar end cap formed into an approximately circular shape has a size larger than a regular polygon opening of the aforementioned barrel.

13. The full-angle coincidence PET detection method according to claim 12, wherein:
when the integrally closed detection cavity has a cylindrical shape, the middle barrel is placed with the axis being horizontal, and the detection cavity has a housing outside; the housing is composed of a barrel housing on an outer surface of the barrel, and end cap housings on outer surfaces of the two planar end caps; each of the two planar end cap housings is connected with the barrel housing by one or more hinges or coupling heads, so as to form an integrally closed detection cavity when closed; moreover, one or more fixation buckle devices are also included for closing the detection cavity;
when the integrally closed detection cavity has a capsule shape, the middle barrel is placed with the axis being horizontal, and the detection cavity has a housing outside; the housing is composed of a barrel housing on an outer surface of the barrel, and end cap housings on outer surfaces of the two concave curved end caps; each of the two concave curved end cap housings is connected with the barrel housing by one or more hinges or coupling heads, so as to form an integrally closed detection cavity when closed; moreover, one or more fixation buckle devices are also included for closing the detection cavity; the concave curved end cap is one of the following three situations: a hemispherical end cap, a less-than-half ellipsoidal end cap or a less-than-half spherical crown-shaped end cap;
when the integrally closed detection cavity has an ellipsoid shape and the barrel is sandwiched in the middle, the middle barrel is placed with the axis being horizontal, and the detection cavity has a housing outside; the housing is composed of a barrel housing on an outer surface of the barrel, and two hemi-ellipsoid housings on outer surfaces of the two left and right hemi-ellipsoids; each of the two hemi-ellipsoid housings is connected with the barrel housing by one or more hinges or coupling heads, so as to form an integrally closed detection cavity when closed; moreover, one or more fixation buckle devices are also included for closing the detection cavity; the barrel sandwiched in the middle of the ellipsoid-shaped detection cavity is a cylindrical barrel or a middle barrel cut from an ellipsoid that satisfies a>b=c;
when the integrally closed detection cavity has an ellipsoid shape and is composed of two upper and lower hemi-ellipsoids or two left and right hemi-ellipsoids, the detection cavity has a housing outside; the housing is composed of two upper and lower hemi-ellipsoid housings or two left and right hemi-ellipsoid housings that fit the two upper and lower hemi-ellipsoids or two left and right hemi-ellipsoids; the two upper and lower hemi-ellipsoid housings or the two left and right hemi-ellipsoid housings are each connected with the barrel housing by one or more hinges or coupling heads, so as to form an integrally closed detection cavity when closed; moreover, one or more fixation buckle devices are also included for closing the detection cavity; and
when the integrally closed detection cavity has a regular polygonal prism shape, the middle barrel is placed with the axis being horizontal, and the detection cavity has a housing outside; the housing is composed of a barrel housing on an outer surface of the barrel, and end cap housings on outer surfaces of the two planar end caps; each of the two end cap housings is connected with the barrel housing by one or more hinges or coupling heads, so as to form an integrally closed detection cavity when closed; moreover, one or more fixation buckle devices are also included for closing the detection cavity.

14. The full-angle coincidence PET detection method according to claim 13, wherein:
a coincidence circuit is connected between every two PET detection modules;
each of the PET detection modules has the following specific structure: a detector housing is wrapped on the outside, a photoelectric sensor array is disposed outwardly, and a PET detection crystal is disposed inwardly; a light guide is disposed between the photoelectric sensor array and the PET detection crystal; the light guide is tightly coupled with both the photoelectric sensor array and the PET detection crystal; and
the material of the PET detection crystal is a scintillation crystal, and the scintillation crystal is composed of one or more crystal blocks.

15. The full-angle coincidence PET detection method according to claim 14, wherein:
the PET detection crystal is selected from one or more of bismuth germanate (BGO) crystals, sodium iodide (NaI) crystals, NaI(Tl) single crystals, lutetium silicate (LSO) crystals, gadolinium silicate (GSO) crystals and yttrium lutetium silicate (LYSO);
the crystal block is specifically a crystal strip array composed of a plurality of crystal strips, or is composed of one or more integrally cut crystals;
spacers made of high atomic number substance are installed between all the detection module rings, or spacers made of high atomic number substance are installed between some of the detection module rings, or no spacers are installed between all the detection module rings; and
the high atomic number substance is lead or tungsten;
the regular polygonal prism is a regular hexagonal prism or a regular octagonal prism, and the regular polygon is a regular hexagon or a regular octagon.

16. The full-angle coincidence PET detection method according to claim 15, wherein:
the crystal strip array is composed of a plurality of crystal strips; and each of the one or more crystal blocks is composed of one or more integrally cut crystals.

17. The full-angle coincidence PET detection method according to claim 16, wherein:
when the integrally closed detection cavity has a capsule shape, the specific configuration of the detection cavity is as follows:
the detection cavity is divided into two left and right halves, and the two left and right halves of the detection cavity have a left support structure and a right support structure respectively for supporting the two left and right halves of the detection cavity;
the two left and right halves of the detection cavity are opened and closed through a linear guide rail located below;
the linear guide rail is a linear guide rail for the movement of a scanning bed, a pad block for adjusting the height of the guide rail is located below the linear guide rail, and a bed assembly above the guide rail can move along the guide rail as a whole; and
the scanning bed can have a scanning bed support, and since the scanning bed support needs a space, part of the PET detection modules can be removed.

18. The full-angle coincidence PET detection method according to claim 17, wherein:
in the step (2), the detection cavity is opened in the form of left and right separation; specifically, the support structures (1) for two left and right halves of the detection cavity drive the two left and right halves of the detection cavity to be separated along the guide rail (2) to the left and right;
placing the detection object in the step (2) is to transfer the detection object to a suitable position on the scanning bed;
closing the detection cavity in the step (3) means that the scanning bed and the scanning bed support (5) move to a scanning position along the scanning bed by means of the linear guide rail (3) and that the two left and right halves of the detection cavity are closed;
in the step (3), the time of flight method is used to screen LORs of the true coincidence events during the calculation; and
after the step (3) is completed, the two left and right halves of the detection cavity are separated along the linear guide rail to the left and right, the scanning bed moves out of the scanning position, the detection object is replaced, and steps (1)-(3) are repeated.
